(19) United States
(12) Patent Application Publication
(10) Pub. No.: US 2011/0165262 A1
(43) Pub. Date: Jul. 7, 2011

(54) REDUCING DRUG DEPENDENCE OR ADDICTION

(75) Inventors: Mishal Hamid Al-Sari, Dubai (AE); Rafida Mohammed Ahamed Al-Dabooni, Dubai (AE); Yousef Jameel, Dubai (AE)

(73) Assignee: VERITRON LIMITED

(21) Appl. No.: 11/814,710

(22) PCT Filed: Jan. 26, 2006
(86) PCT No.: PCT/GB2006/000260

§ 371 Date: Jan. 21, 2009

(30) Foreign Application Priority Data
Jan. 26, 2005 (GB) 0501655.5

(51) Int. Cl. A61K 33/24; A61K 36/00; A61K 36/28; A61K 38/28; A61K 38/27; A61K 31/51; A61K 31/34; A61P 25/30; A61P 25/32; A61P 25/34

(52) U.S. Cl. 424/617; 424/725; 424/764; 514/5.9; 514/11.3; 514/276; 514/474

(57) ABSTRACT

Drug dependence or drug addiction is reduced by the administration to a subject of one or more of the following components: an antioxidant; vitamin B; a metal salt that provides metal ions, in vivo; insulin or a growth hormone; an antihistamine; and a herbal extract.

### FIELD OF THE INVENTION

This invention relates to a new use of therapeutic agents, for reducing drug dependence or addiction.

### BACKGROUND OF THE INVENTION

The abuse, overuse or misuse of drugs, e.g. drugs of dependence, including narcotics, hallucinogens, opiates, alcohol and nicotine, presents problems of various types in different societies. Treatment or rehabilitation of addicts in particular is desirable, but has not been achieved effectively and/or without disturbance to the patient. It is also desirable to reduce the subject's need for such drugs, and/or the amount taken or given.

Various components which, in combination, are useful in therapy, especially the treatment of tumours, are described in WO03/101479 which is hereby incorporated in full, by reference. The possible role of each component is suggested. For example, it is suggested that cell permeability may be increased.

### SUMMARY OF THE INVENTION

It has now been found that one or more components of the composition described in WO03/101479 leads to improved efficacy, or potentiation, of drugs. Thus, whatever effect the drug is intended to have, the amount that is required to achieve that effect may be reduced. This means that dependence on or addiction to a drug can be reduced, and rehabilitation may be achieved.

### DESCRIPTION OF THE INVENTION

Any drug of abuse may be “used” in this invention. Examples are given above, and others will be apparent to those skilled in the art. They may be taken by the user, e.g. for recreational purposes, examples being alcohol, nicotine, steroids, performance-enhancers, hallucinogens etc, or administered in the course of therapy, examples being opioids.

The one or more components used in the invention may be administered together with or separately from the drug of abuse, and by the same or different route. These components will now be described in more detail, but it must be understood that only one, or a combination of two, three, four or more may be required. For example, insulin and/or an antihistamine may be useful, with or without other components.

One component for use in the invention is an antioxidant. The function of this component may be to prevent the formation of S—S bridges by oxidation of cysteine residues. Disulfide linkages are caused by many oxidising agents, and cause loss of enzymatic activity. Alternatively or in addition, the antioxidant may inhibit the production of oxygen radicals (free radicals) as a by-product of the normal metabolism of oxygen. These oxygen radicals are very damaging to cell membranes, proteins, lipids and DNA. Oxidative damage accumulates with age and is considered to be a major contributor to ageing and the development of degenerative diseases (e.g. cancer, cardiovascular disease, immune system decline, etc).

Suitable antioxidants for use in the invention are small molecules such as vitamin C, A and E. It will be appreciated that a suitable precursor of any such compound may be used, e.g. β-carotene. The preferred antioxidant for use in this invention is vitamin C, e.g. as Sodium ascorbate or ascorbic acid. A suitable dosage of this component is 1 to 500 mg/kg/day.

Alternatively or in addition, this component may comprise one or more components of vitamin B. Many enzymes catalyse reaction of their substrates only in the presence of a specific non-protein molecule, i.e. a coenzyme. Coenzymes frequently contain B vitamins as part of their structure. One or more of vitamins B1 (thiamine hydrochloride), B2 (riboflavin sodium phosphate), PP (nicotinamide), B6 (pyridoxine hydrochloride) and B5 (dexpanthenol) may be used. The amount of each such component is, for example, 0.1 to 50 mg/kg/day.

A second component for use according to the present invention is a metal salt that provides metal ions, in vivo. The nature of the anion is not critical, and will generally be chosen to be non-toxic and of suitable solubility or other appropriate compatibility with other components of the medicament. Many metal ions act as positive modifiers, and certain enzymes require the presence of metal ion for full activity. The function of the metal ion may be to complement the coenzyme. The ion may be, for example, Na, K or multivalent such as Fe, Mo, Mg, Mn, Ca, Zn, Cu or Co. This may be in the form of a salt, of which many examples are known, e.g. with any inorganic acid such as HCl or H₂SO₄, or an organic acid such as acetic, ascorbic, citric, gluconic, glutamic, maleic, malic or succinic acid. A preferred component of this type is Calcium Gluceptate. A typical dosage of this component is 1 to 1000 mg/kg/day.

A third component for use according to the invention may be an agent that increases the permeability of cell membranes or otherwise enhances transport, e.g. by action on receptors. This material may enhance the ability of the cells that need treatment to receive the other active material or materials that may be included in the novel medicament, especially in human therapy (it may not be required in veterinary medicine). A preferred agent of this type is insulin or a growth hormone. A typical dosage of insulin is 1 to 1000 IU/kg/day.

Another component is an antihistamine. Such a material can not only prevent or reduce abnormal reactions, especially allergic reaction, but also prevent the accumulation of substances which block transport, e.g. by binding to cell membrane receptors.

As is known to those of ordinary skill in the art, antihistamines work by competing with histamine released by mast cells and basophils for histamine receptors on the mucosa of the eyes, nose, bronchial airways and skin. The antihistamine binds to the receptor and prevents histamine attachment, thereby blocking the effect of histamine in the tissues. Antihistamine drugs counteract the physiological effects of histamine production, in allergic reactions and colds.

Antihistamines can be divided into classical and non-sedating antihistamines. There are many examples of such compounds, including acrivastine, azatadine, azelastine, brompheniramine, carbinoxamine, cetirizine, chlorpheniramine, clemastine fumarate, cyproheptadine, diphenhydramine, doxepin, hydroxyzine, fexofenadine, loratadine, meclizine, phenindamine, promethazine, pyrilamine and tripolidine.

A preferred material for use in the invention is chlorpheniramine maleate. A suitable dosage of such a component is 0.1 to 50 mg/kg/day.

Yet another component is a herbal extract. It has been found that desirable effects can be provided by the use of natural substances. The available evidence suggests that an extract of such herbal substances obtained as described below may cause an increase in the level of the cytokine IL-6. This is a good measure of the desirable properties of this extract. Other cytokines that may be affected, in order to obtain the desirable properties of the invention, are other interleukins such as IL-7, IL-8, IL-9 and IL-10, and also tumour necrosis factor (TNF).

The extract may be obtained by any suitable procedure, including methods known to those of ordinary skill in the art. The extract may be obtained by using an aqueous or organic medium, and separated from other components by filtration, chromatography etc. For example, a material that may be used in the invention is derived from the dried flower heads of the composite plant Matricaria chamomilla, the seeds of the plant Nigella Sativa, the gum of the plant Acacia Senegal, or one or more materials therein, include polysaccharides, glycoproteins, volatile oils, azolene, anthemic acid, apogenin, glycosides and other substances. The material may be water-soluble.

It is also believed that certain types of honey may comprise components that provide an activity that is analogous to that obtained from the components of the plants described above.

Other active materials may also be given to the subject. Although it is not believed that further materials are necessary, it has been found that certain steroids and vitamins, typically given orally, can support or enhance the effect of the medicament. Suitable steroid hormones may increase the synthesis of specific proteins, by unmasking certain cistrons, with the assistance of essential metabolites such as vitamins and amino acids. Examples of suitable steroids are estradiol, nandrolone and estriol. Vitamins such as A, D and/or E may also be given. The function of vitamin A may be to preserve the integrity of epithelial tissue, to play a role in protein synthesis, and to stabilise cell membranes and also subcellular membranes.

As is conventional in medicine, a medicament for use in the invention may comprise other components, depending on the intended effect, the nature of the formulation, the route of administration, and other factors that are known to those skilled in the art. Thus, for example, the medicament may be formulated in water, e.g. to provide an aqueous solution or suspension suitable for injection. It may be desirable to include in any such formulation one or more additional substances that aid dissolution or suspension of active components, such as an organic or a polar solvent. The composition may comprise conventional excipients, for example, phenol (which acts as a preservative).

Compositions for use according to the invention can be formulated by methods known to those skilled in the art. Pharmaceutically acceptable components should be used. The term “pharmaceutically acceptable” refers to those properties and/or substances which are acceptable to the patient from a pharmacological/toxicological point of view and to the manufacturing pharmaceutical chemist from a physical/chemical point of view regarding factors such as formulation, stability, patient acceptance and bioavailability.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients such as, for example, inert diluents such as calcium carbonate, sodium carbonate, lactose, calcium phosphate or sodium phosphate; granulating and disintegrating agents, for example corn starch or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated, to form osmotic therapeutic tablets for control release.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

The composition may be an aqueous solution or suspension. It may contain the active materials in admixture with suitable excipients. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents, for example a naturally occurring phosphatide such as lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol, such as a polyoxyethylene with partial esters derived from fatty acids and hexitol anhydrides, for example polyoxyethylene sorbitan monooleate. Aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents (such as those set forth above) and flavouring agents may be added to provide a palatable oral preparation. These compositions may be preserved by the addition of an anti-oxidant such as Ascorbic Acid or Sodium ascorbate or ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified above. Sweetening, flavouring and colouring agents may also be present.

A pharmaceutical composition for use in the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated using suitable dispersing or wetting agents and suspending agents, examples of which have been mentioned above. A sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition may also be administered in the form of suppositories for rectal administration of the drug. Such compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, suitable compositions are in the form of, for example, creams, ointments, jellies, solutions or suspensions. For the purposes of this specification, topical application includes mouth washes and gargles.

As indicated above, composition of the invention may be given by injection. Intramuscular injection is preferred, although any parenteral administration is suitable.

It may also be preferred that the composition is given orally, although insulin should not usually be included in an oral formulation. Oral administration may be particularly preferred for veterinary medicine.

Although some indication has been given as to suitable dosages of certain materials, the exact dosage and frequency of administration depend on several factors. These factors include the particular components that are used, the particular condition being treated, the severity of the condition, the age, weight and general physical condition of the particular patient, and other medication the individual may be taking, as is well known to those skilled in the art.

The following Examples illustrate the invention.

### Example 1

### Medicament

The following substances were formulated:

| | | | |
|---|---|---|---|
| 1. | Dried flower heads of the composite plant | 360 | mg/ml |
| | Matricaria chamomile | | |
| 2. | Pyrogen-free distilled water | 2.0 | Liter |
| 3. | Phenol | 2.64 | mg/ml |
| 4. | Sodium ascorbate or ascorbic acid (Vitamin C) | 20.2 | mg/ml |
| 5. | Calcium Gluceptate (ionized calcium) | 39.7 | mg/ml |
| 6. | (Insulin) a neutral solution of biosynthetic | 0.088 | mg/ml |
| | insulin | | |
| 7. | Chlorpheniramine Maleate | 0.838 | mg/ml |
| 8. | Vitamin B1 (thiamine hydrochloride) | 0.838 | mg/ml |
| 9. | Vitamin B2 (riboflavin sodium phosphate) | 0.458 | mg/ml |
| 10. | Vitamin PP (nicotinamide) | 3.352 | mg/ml |
| 11. | Vitamin B6 (pyridoxine hydrochloride) | 0.335 | mg/ml |
| 12. | Vitamin B5 (dexpanthenol) | 0.503 | mg/ml |

Two liters of pyrogen-free distilled water were poured into a large beaker that was previously cleaned and washed with pyrogen-free distilled water and sterilized in an oven at 125° C. for one hour.

The dried flower heads of the composite plant Matricaria Chamomile were washed thoroughly with cold pyrogen-free distilled water. The washed flower heads were added to the water in the beaker and heated and stirred until the temperature reached 95° C.

When the temperature was close to 35° C., the contents of the beaker were filtered through three layers of filter paper (previously washed with absolute ethanol). The filtrate was collected in another sterile pyrogen-free beaker. Calcium Gluceptate and Sodium ascorbate or ascorbic acid (Vitamin C) were added to the beaker and stirred. Again, the contents of the beaker were then filtered through three layers of filter paper (which was previously washed with absolute ethanol). The 7.5 ml of carbolic acid, preheated to 65° C. (its melting point is 63° C.) was added to the contents of the beaker and stirred well. When the temperature was close to 10° C., the vitamins B1, B2, PP, B6 and B5 (Becozyme), chlorpheniramine maleate (Allerfin) and neutral solution of biosynthetic insulin were added to the contents of the beaker and stirred well.

The resultant medicament was poured into small vials or ampoules of 5.0 ml or large vials or bottles of 100 ml or 50 ml of the type used for intravenous fluids. This medicament was suitable for use as an injectable.

A preferred process for preparing a chamomile extract is as follows:

Chamomile flowers of suitable quality, i.e. not too dry, are gently pressed through a 850 mm screen to remove dust and pick out green leaves. After discarding dust and particles, the flower heads retained by the screen are rubbed between the hands to release the yellow particles from the heads. The particles are then passed through a 850 mm screen to remove large bits of unwanted flower heads, and screened again three times through a 850 mm screen, shaking vigorously from side to side, to leave behind the lighter unwanted fraction. The resulting fraction is yellow in colour and slightly shiny.

To 100 g of the fraction is added approximately 2 litres of pyrogen-free sterile water for injection in a 3-litre clean glass beaker, and mixed by stirring. The product is heated using a hot plate to 95° C., then allowed to cool to approximately 35° C. The extraction mixture is sieved through a 355 mm screen to remove particles. The filtrate is passed through a 0.2 μm sterile filter, to give the desired liquid chamomile extract.

### Therapeutic Examples

The medicament described above was given as a 5 ml dosage, intramuscularly, twice daily (5 ml every 12 hours) for an average body weight of 75 kilograms (subjects 50-100 kg body weight). The dose can be calculated, according to the body weight, as 0.072 ml/kg body weight twice daily.

Estradiol (5 mg) and Vitamin A (40000 IU) were given separately every week. In addition, estriol (0.5 mg) was given separately to the patient every day. Finally, nandrolone decanoate (Deca durabolin) (25 mg) was given separately to the patient every 20 days.

In various healthy animal and human models, this regimen has proved to be safe. Therapy according to the present invention has been successful in reducing nicotine craving in a patient.

1. A method for reducing drug dependence or drug addiction in a subject, which comprises the administration to the subject of one or more of the following components: an antioxidant; vitamin B; a metal salt that provides metal ions, in vivo; insulin or a growth hormone; an antihistamine; and a herbal extract.

2. A method according to claim 1, wherein the one or more components comprise the antioxidant and the metal salt.

3. A method according to claim 2, wherein the antioxidant is ascorbate.

4. A method according to claim 1, wherein the one or more components comprise the antihistamine and/or insulin.

5. A method according to claim 1, wherein the one or more components comprise one or more active compounds of chamomile.

6. A method according to claim 1, wherein the one or more components comprise one or more active components of the plant Nigella Sativa.

7. A method according to claim 1, wherein the one or more components comprise one or more active components of the plant Acacia Senegal.

8. A method according to claim 1, wherein the one or more components are in the form of an injectable formulation.

9. A method according to claim 1, wherein the subject is taking a drug of dependence.

10. A method according to claim 1, wherein the subject is taking a drug of abuse.

11. A method according to claim 10, for rehabilitation.

12. A method according to claim 1, wherein the subject is taking a alcohol or nicotine.

13. A method according to claim 12, for reducing the craving for nicotine.

14. Use of one or more components as defined in claim 1, for the manufacture of a medicament for use in reducing drug dependence or drug addiction.

15. A method according to claim 4, wherein the one or more components comprise one or more active compounds of chamomile.

16. A method according to claim 4, wherein the one or more components comprise one or more active components of the plant Nigella Sativa.

17. A method according to claim 4, wherein the one or more components comprise one or more active components of the plant Acacia Senegal.

18. A method according to claim 15, wherein the one or more components are in the form of an injectable formulation and the subject is taking a drug of abuse.

19. A method according to claim 16, wherein the one or more components are in the form of an injectable formulation and the subject is taking a drug of abuse.

20. A method according to claim 17, wherein the one or more components are in the form of an injectable formulation and the subject is taking a drug of abuse.
